(19) United States
(12) Patent Application Publication
(10) Pub. No.: US 2005/0089520 A1
(43) Pub. Date: Apr. 28, 2005

(54) Antibody stimulating il-ira production

(76) Inventors: Dominique Bourel, Germaine (FR); Martine Bruley-Rosset, Bonneuil (FR); Frederic Dhainaut, Dourdan (FR); Jacky Lirochon, Vignes (FR)

Correspondence Address: FOLEY AND LARDNER;SUITE 500, 3000 K STREET NW, WASHINGTON, DC, 20007, US

(21) Appl. No.: 10/494,964

(22) PCT Filed: Nov. 8, 2002
(86) PCT No.: PCT/FR02/03854

(30) Foreign Application Priority Data
Nov. 9, 2001 (FR) 01/14518

(51) Int. Cl. A61K 39/395; C07K 16/18; C12N 5/06

(52) U.S. Cl. 424/145.1; 530/388.25; 435/69.1; 435/320.1; 435/326

(57) ABSTRACT

The invention concerns natural antibodies eventually monoclonal capable of inducing a high production of IL-1Ra with monocytes or macrophages and the use of said antibodies for treating inflammatory or autoimmune diseases.

The present invention relates to the use of natural IgG-class antibodies capable of inducing a high production of IL-1Ra by monocytes or macrophages and to the use of said antibodies for treating inflammatory or autoimmune diseases.

IL-1 is a cytokine corresponding to two proteins (IL-1a and IL-1b) which play a key role in the inflammatory response (Dinarello C A, Blood, 1996, 87: 2095, 2137). Although IL-1 is considered to be the product of activated macrophages, it is also synthesized by lymphoid and non-lymphoid cells such as vascular cells, epithelial cells, skin cells, nerve cells, dendritic cells, fibroblasts and chondrocytes. It is considered to be a central mediator of the host's response to infection and to other forms of trauma.

IL-1 is a multifunctional cytokine which acts on a large variety of cells of the immune system, but also other cell systems. IL-1 acts directly on the central nervous system, induces the inhibition of insulin secretion by pancreatic islet cells, and stimulates cellular, humoral and natural immune responses.

IL-1α and IL-1β act by binding to surface receptors expressed on many cells and trigger a series of signals having effects on cell proliferation, apoptosis, cytokine induction, prostaglandin production, receptor activation and the secretion of degradation enzymes. Two receptors exist, which interact with IL-1a and IL-1b: the type I receptor (80Kd) expressed on T cells, fibroblasts and epithelial cells, which requires interaction with IL1RacP (IL-1 accessory protein) in order to transmit the signal, and the type II receptor (67Kd) expressed on B cells, neutrophils and macrophages, which cannot transmit the signal because it lacks the intracellular portion.

A third member of the Il-1 family, IL-1Ra (IL-1 receptor antagonist), acts as a natural antagonist of IL-1a and IL-1b and interacts with the type I IL-1 receptor but does not transduce the intracellular signal or the biological response. These three proteins have 25 to 30% of the amino acids in common. IL-1Ra associates poorly with the type II receptor.

IL-1 is considered to be a mediator of the host's defense against infections, but also many pathologies, and a high circulating IL-1 level correlates with a variety of clinical situations, and in particular septic shock. Its activity is finely regulated at the level of transcription, translation and secretion. Other regulatory mechanisms involve the action of other cytokines, the difference in expression of IL-1 receptors and the production of natural inhibitor such as IL-1Ra.

Many experimental studies and some clinical trials have shown the protective role of IL-1Ra in a certain number of pathologies: septic shock, rheumatoid arthritis, transplant rejection, and heart attack and ischemia.

IL-1Ra can act as an IL-1 competitor and therefore represents an advantageous therapeutic agent. WO 95/16706, WO 99/36541, WO 01/42305, WO 01/41792 and WO 01/42304 describe the IL-1Ra peptide, mutated peptides, and their use for treating various pathologies.

However, while therapeutic trials using soluble recombinant IL-1Ra have shown good efficacy for this product in septic shock, rheumatoid arthritis and GVH, the very high doses required and the short half-life of recombinant IL-1Ra are obstacles to its use.

Consequently, any strategy aimed at specifically stimulating the production of IL-1Ra and therefore at inhibiting the inflammatory activity of IL-1 is of therapeutic interest in the treatment of a certain number of acute or chronic inflammatory pathologies such as rheumatoid arthritis, GVH and the like.

The present invention relates to the use of antibodies capable of strongly stimulating the in vitro (and in vivo) production of IL-1Ra (interleukin 1 receptor antagonist), but not that of a pro-inflammatory cytokine, IL-1b (interleukin 1b and 1a). These IgG-type human antibodies have the characteristic of recognizing, in ELISA's, self proteins such as actin, myosin, tubulin, DNA or myelin basic protein (MBP), but not vaccine antigens such as tetanus toxoid. They are also selected by virtue of their ability to bind to the hapten dinitrophenyl or DNP.

These antibodies can therefore substitute advantageously for IL-1Ra for any therapeutic applications, and correspond to the problems mentioned above.

### DESCRIPTION

Thus, in a first aspect, the invention relates to natural IgG-class antibodies which react with the hapten DNP and at least one autoantigen selected from myosin, actin, myelin basic protein (MBP) and tubulin, characterized in that they induce overexpression of IL-1Ra at the mRNA and secreted protein levels.

The term “overexpression” means that the level of expression of IL-1Ra is at least 50% greater than the level of expression of the cells not stimulated with the antibodies of the invention.

The expression “natural IgG-class antibodies” is intended to denote antibodies, antibody fractions or antibody subpopulations or monoclonal antibodies exhibiting reactivity with respect to self antigens. The IgG subpopulations are prepared from polyvalent Igs or any other intermediate fraction obtained during the process for producing IVIgs for therapeutic use.

Advantageously, such natural antibodies do not react with tetanus toxoid or with other non-self antigens.

More specifically, they exhibit a reactivity with respect to the hapten DNP which is greater than 100, 125 or 150 relative to the IVIgs, in particular relative to Tegeline®. In addition, these antibodies exhibit a degree of reactivity with respect to at least one autoantigen selected from DNA, myosin, MBP, actin and tubulin, which is greater than 50, 75 or 100 relative to the polyvalent IVIgs, in particular relative to Tegeline®. The degree of enrichment can be calculated relative to the activity of the initial polyvalent Igs as control value in the case of IgG subpopulations, or relative to a reference monoclonal antibody when the natural antibody is a monoclonal antibody.

In a second aspect, the invention relates to a method for preparing an IgG subpopulation defined above, characterized in that it comprises the following steps:
a) preparing an insoluble support onto which DNP-lysine is grafted,
b) adsorbing polyvalent Igs onto the support obtained in step a),
c) eluting the Igs retained on the support so as to collect the fraction which interacts with DNP,
d) selecting the fractions which exhibit reactivity with respect to given autoantigens, and
e) selecting the fractions which induce overexpression of IL-1Ra by macrophages or monocytes.

The general method for preparing polyvalent Igs is known to those skilled in the art and comprises essentially the following steps:
fractionation of the plasma originating from a pool of donors by precipitation, adsorption and filtration then ultrafiltration (obtaining a first fraction “PSO 1”),
treatment with pepsin at acid pH, formulation, distribution and lyophilization (obtaining the product Tegeline®),
   of course, the polyvalent Igs can be prepared using other methods.

Currently, the tolerance and the efficacy of the polyvalent IgGs which have been marketed, in particular Tegeline® (LFB, France), are recognized in particular in the treatment of ITP, of Kawasaki disease and of Birdshot retinochoroiditis, pathologies for which marketing authorizations have been obtained.

In the context of the invention, the term “polyvalent Igs” is intended to mean whole polyvalent IgGs, polyvalent IgG fragments such as F(ab′)2 or F(ab), and any intermediate fractions obtained during the process for producing polyvalent IVIgs.

Step a) can be carried out by grafting DMP-lysine onto a solid support, in particular onto a Sepharose®, Trisacryl®, Affiprep® or Affigel® gel, gels activated with CNBr, NHS or C₅H₈O₂ (glutaraldehyde). Thus, in the method, the IgGs deposited onto the solid support obtained in step a) are adsorbed either in the form of polyvalent IgG dissolved after lyophilization, or of polyvalent IgG in liquid form, or in the form of intermediate fractions obtained during a process for producing polyvalent IgGs in phosphate buffer comprising NaCl, the concentration of which can range from 0M to 3M.

The IgGs retained in step b) are eluted with a buffer of ions dissociating the bond between the IgGs and the DNP, selected in particular from chaotropes such as glycine-HCl or sodium iodide (NaI), under conditions varying the pH and/or the molarity of the buffer.

Step d) comprises an ELISA assay carried out on a panel of autoantigens selected in particular from DNA, actin, myosin, MBP and tubulin or any other self antigen. This step may also comprise measuring the reactivity for a vaccine antigen, for example tetanus toxoid, taking the degree of enrichment relative to the activity of the initial polyvalent Igs as control value.

Next, in step e), subpopulations are selected which have the properties mentioned above, i.e. subpopulations which induce overexpression of the IL-1Ra mRNA and protein by macrophages or monocytes. It is considered that there is overexpression when a significant increase in expression is detected compared to the baseline expression observed naturally in macrophages or monocytes (for example an increase greater than 50% or more (for example 100% or 200%)).

The invention also relates to the subpopulations which can be obtained using this method.

The invention also relates to a method for preparing monoclonal antibodies defined above, characterized in that it comprises the following steps:
a) transforming B lymphocytes originating from the blood of a normal donor, with EBV,
b) identifying clones, in each culture well, secreting IgGs capable of recognizing the hapten DNP and at least one given autoantigen, using an ELISA assay,
c) amplifying the clones identified in step b),
d) purifying the antibodies produced by the clones of step c), and
e) selecting the monoclonal antibodies which induce overexpression of IL-1Ra by human macrophages or monocytes.

To improve the stability of expression of the natural Igs, the clones identified in step b) can be fused with a non-Ig-secreting myeloma.

In this method, the autoantigen can be selected from DNA, actin, myosin, myelin basic protein (MBP) and tubulin.

In another aspect, the invention is directed toward the selected clones of EBV-transformed B cells, possibly fused with a myeloma, and amplified by the method mentioned above. These clones are characterized in that they produce natural monoclonal antibodies capable of inducing overexpression of IL-1Ra by macrophages or monocytes.

The invention also relates to the monoclonal antibodies obtained with said clones. Such antibodies are capable of inducing overexpression of IL-1Ra by macrophages or monocytes.

In an additional embodiment, the invention relates to a composition comprising a natural antibody defined above (antibody subpopulation or monoclonal antibody) and a pharmaceutically acceptable vehicle. Advantageously, this composition is suitable for intravenous, subcutaneous or intramuscular administration.

The invention is also directed toward the use of a natural antibody mentioned above, for preparing a medicinal product. Preferably, this medicinal product is intended for treating and preventing inflammatory diseases. It may be useful for treating and preventing septicemia and septic shock, and for treating and preventing arthritis, rheumatoid arthritis, polyarthritis, osteoporosis, inflammatory bowel diseases, autoimmune diseases, transplant rejection, ischemia, traumas, in particular brain traumas, psoriasis, dermatoses, restenoses, respiratory diseases, allergic rhinitis, allergies and cancer.

### LEGENDS

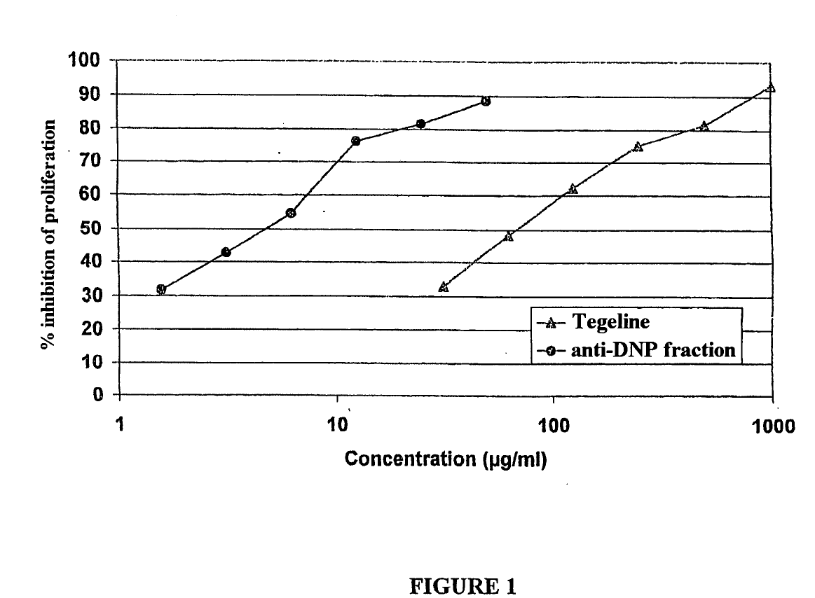

FIG. 1: Inhibitory capacity of Tegeline® and of the anti-DNP fraction on the proliferation of human lymphocytes stimulated in mixed lymphocyte culture (MLC).

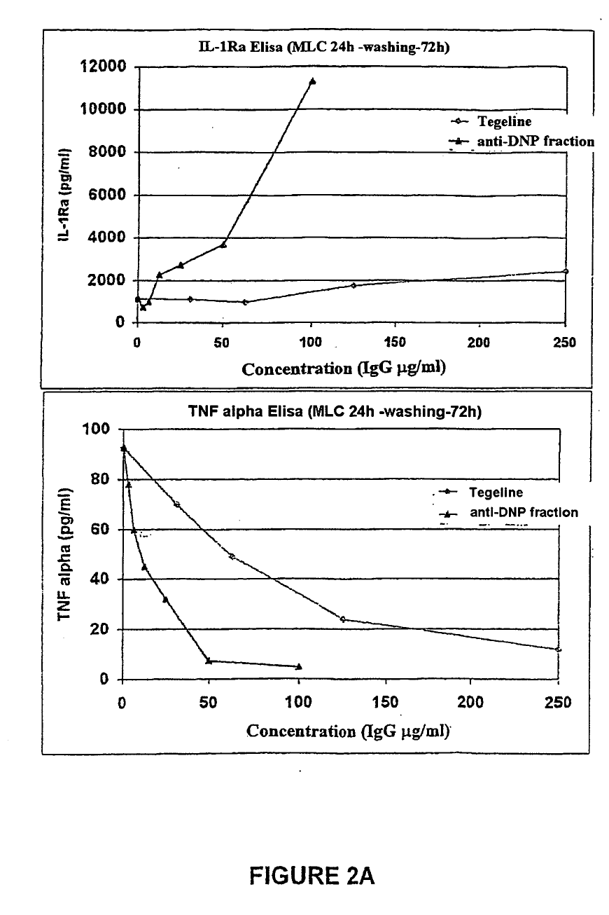

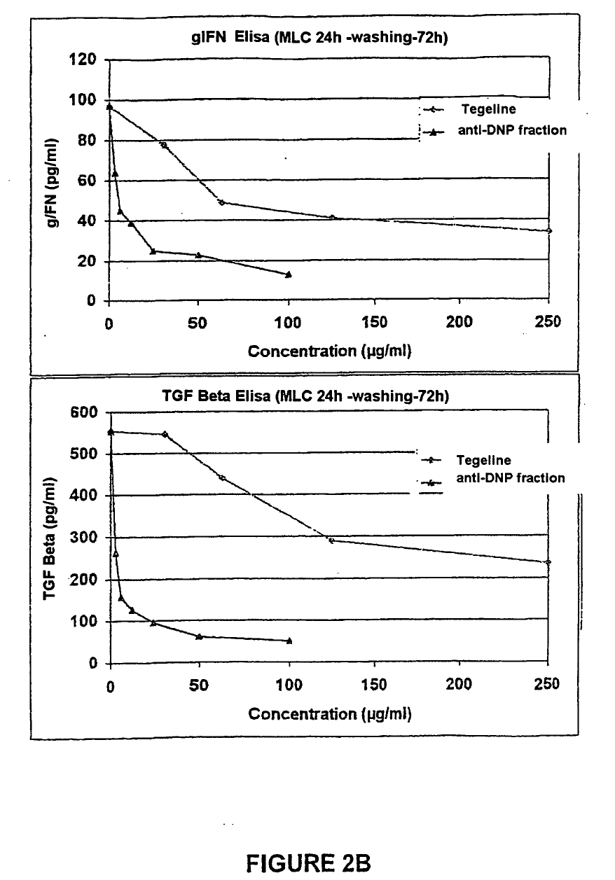

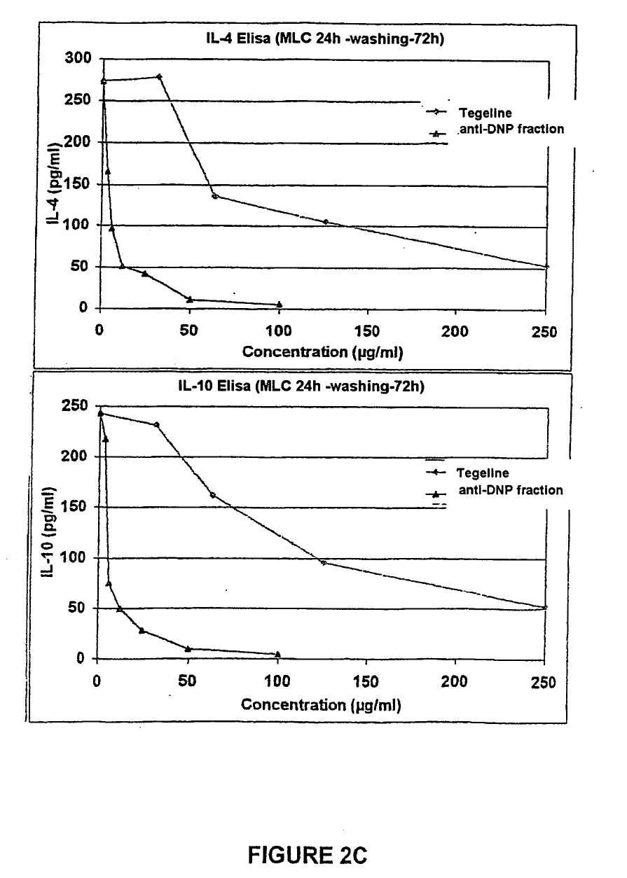

FIGS. 2A-B-C: Effect of Tegeline® and of the anti-DNP fraction on the production of cytokines by human PBLs stimulated in MLC.

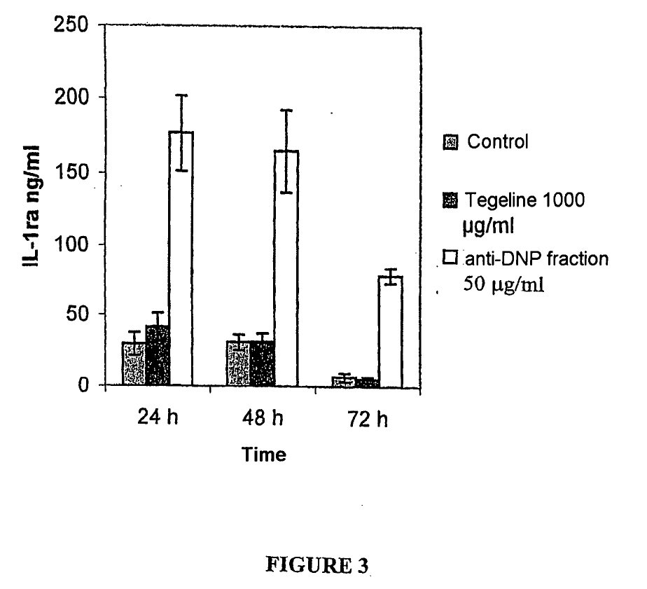

FIG. 3: Secretion of IL1Ra by mouse macrophages in vitro.

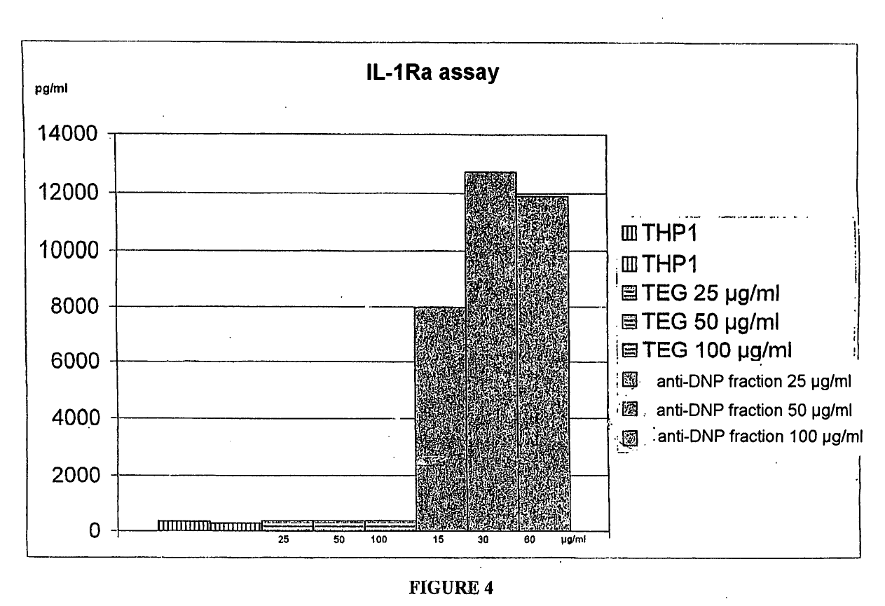

FIG. 4: Production of IL-1Ra by THP1 cells stimulated with various doses of Tegeline® or of the anti-DNP fraction.

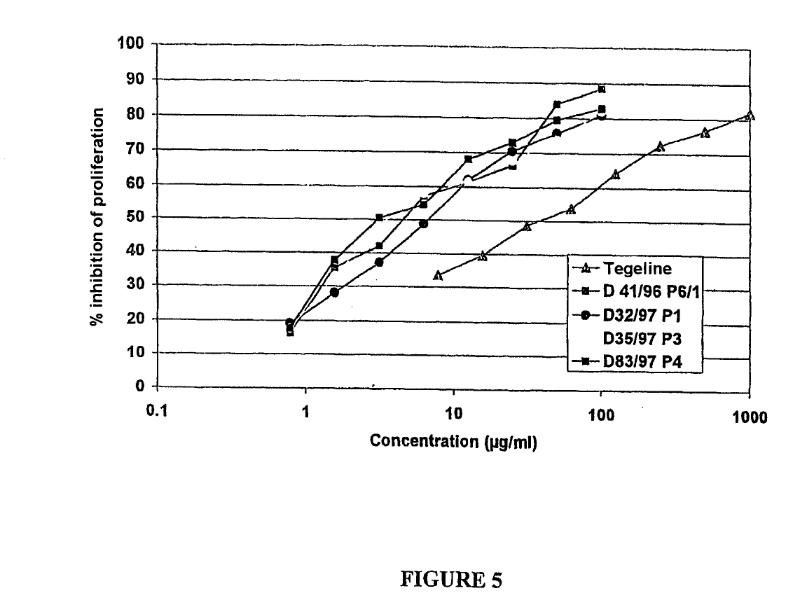

FIG. 5: Inhibitory capacity of Tegeline® and of anti-D monoclonal antibodies on the proliferation of human lymphocytes stimulated in MLC.

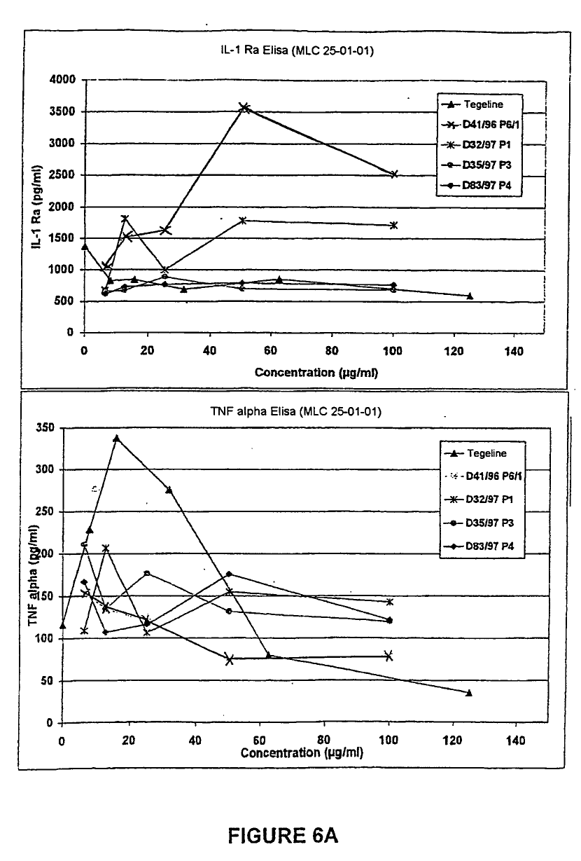

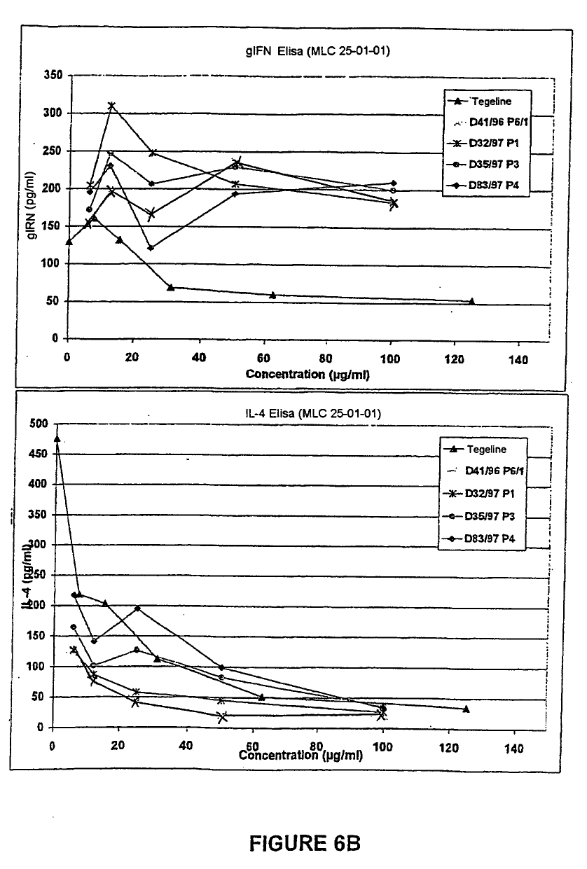

FIGS. 6A-B: Effect of Tegeline® and of anti-D monoclonal antibodies on the production of cytokines by human PBLs stimulated in MLC.

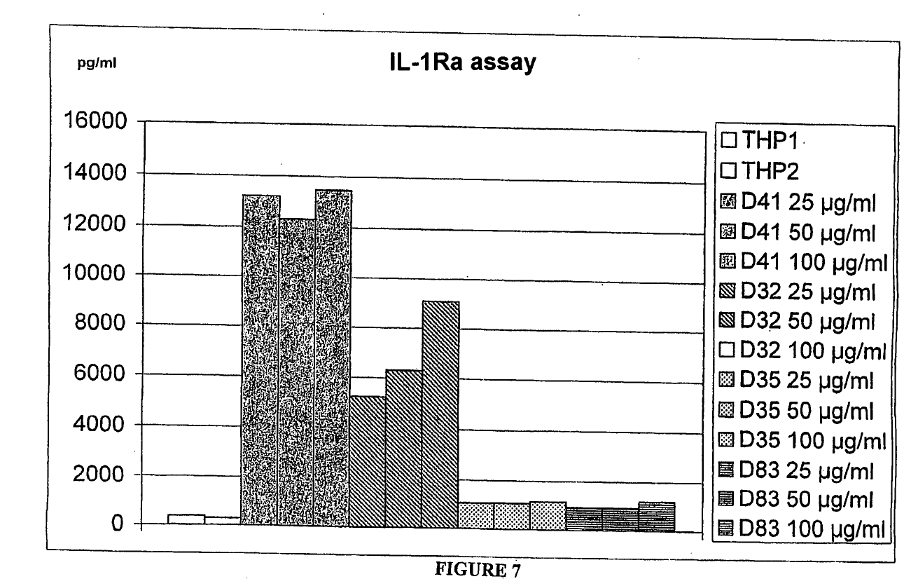

FIG. 7: Production of IL-1Ra by THPA cells stimulated with various doses (25, 50, 100 μg/ml) of anti-D monoclonal antibodies

| Monoclonal antibodies | Anti-DNP |
|---|---|
| D41/96 | (+++) |
| D32/97 | (+) |
| D35/97 | (−) |
| D83/97 | (−) |

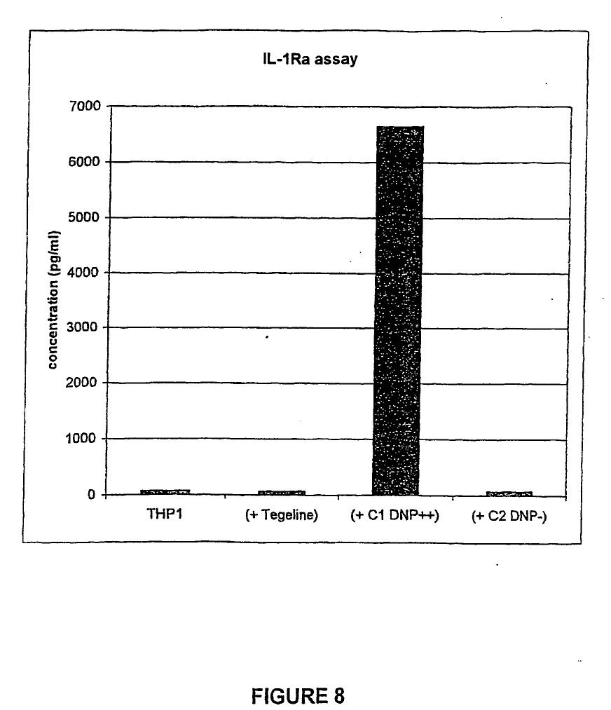

FIG. 8: ELISA assay of IL-1Ra in the culture supernatant from THP1 cells stimulated with 25 μg/ml of Tegeline® or with IgG originating from C1(DNP++) EBV+ B cells or C2(DNP−) EBV+ B cells.

### Example 1

### Obtaining an Anti-DNP IgG Fraction From Polyclonal Immunoglobulins Derived From a Pool of Plasma Originating From Normal Donors (IVIgs)

It has previously been shown (FR 99/16632) that passing a preparation of IVIg (Tegeline®, LFB) over an immunoadsorbent of NHS-Affiprep coupled to DNP-lysine makes it possible to obtain, by elution with sodium iodide (NaI), an eluate representing 0.13% of the IgGs loaded. This eluate has strong reactivity, by ELISA, for DNP-albumin and for many autoantigens such as actin, myosin, MBP, DNA and tubulin, but not tetanus toxoid (table 1).

TABLE 1
Reactivity by ELISA of the anti-DNP fraction
relative to Tegeline ® with respect to several antigens
Degree of enrichment of the anti-DNP fraction relative to Tegeline ®

| Tetanus | | | | | | | |
|---|---|---|---|---|---|---|---|
| toxoid | F(ab′)2 | DNP | Actin | Myosin | MBP | DNA | Tubulin |
| 4.8 ± 2.2 | 4.5 ± 1.3 | 235 ± 100 | 158 ± 64 | 110 ± 33 | 94 ± 66 | 26.0 ± 17 | 238 ± 108 |

This anti-DNP fraction is capable of inhibiting ten to twenty times more than the starting IgGs (Tegeline®) the proliferation of two suspensions of human PBLs that are incompatible in MLC (FIG. 1).

ELISA analysis of the presence of several cytokines in the supernatant of the MLCs shows that the secretion of gIFN, of IL-4, of IL-10, of TNFa and of TGFb is more strongly inhibited by the anti-DNP fraction than by Tegeline®. On the other hand, the anti-DNP fraction very strongly stimulates the synthesis of IL-1Ra (FIG. 2).

Incubation of 2×10⁶ mouse peritoneal macrophages in vitro with the anti-DNP fraction for 24 h, 48 h and 72 h causes the release of mouse IL-1Ra; on the other hand, incubation with Tegeline® has no effect (FIG. 3).

The amount of IL-1Ra mRNA in the THP1 cells (human monocyte cell lines) was measured by quantitative PCR. The level of expression of the IL-1Ra mRNA compared to the level of expression of actin mRNA is greatly increased after incubation of the THP1 cells in the presence of the anti-DNP fraction, but not in the presence of Tegeline® (table 2). The expression of TNFα mRNA is also increased in the presence of the fraction, but to a lesser degree.

TABLE 2
Effect of Tegeline ® and of the anti-DNP
fraction on the synthesis of TNFα mRNA and of IL-1Ra
mRNA by the THP1 cell

| Samples (assay by | | |
|---|---|---|
| quantitative | TNFα mRNA/β-actin | IL-1Ra mRNA/β- |
| RT-PCR) | mRNA | actin mRNA |
| THP1 cells | 4 | 2 |
| THP1 cells (+Tegeline ® | 1 | 9 |
| 50 μg/ml) | | |
| THP1 cells (+anti- | 21 | 148 |
| DNP fraction | | |
| 50 μg/ml) | | |

Similarly, the supernatant from the THP1 cells stimulated with the anti-DNP fraction contains a considerable amount of IL1-Ra, whereas, at the same concentrations, Tegeline® does not induce any production of this cytokine (FIG. 4).

Previous experiments (patent no. 99 16632) had demonstrated that this anti-DNP fraction administered in vivo protected against the development of polyarthritis induced by collagen and of experimental autoimmune encephalomyelitis (EAE) induced by MBP in rats, and of diabetes in NOD mice.

This protective activity is greater after injection of anti-DNP IgGs compared to the unfractionated IVIgs. These natural antibodies could represent an effective treatment for certain inflammatory pathologies.

### Example 2

### Obtaining Natural Monoclonal Human Antibodies Having the Property of Stimulating IL1-Ra Production

### Example 2.1

### Production of Monoclonal Antibodies From Human Peripheral Blood Lymphocytes Originating From an Immunized Donor

A series of human monoclonal antibodies (Mabs) was obtained after transformation with the EBV virus of B lymphocytes originating from the blood of donors immunized with Rh+ red blood cells. These antibodies are all of IgG1 type and have the property of agglutinating Rh+ red blood cells. They are therefore specific for the D antigen.

Fourteen anti-D Mabs were tested for their ability to recognize, in an ELISA, the hapten DNP and the autoantigens described above.

Out of the fourteen Mabs, only two Mabs, D41 and D32, recognize DNP, but only D41 binds to the autoantigens but not to tetanus toxoid. The other Mabs recognize none of the antigens to a greater extent than Tegeline® (table 3).

TABLE 3
Reactivity by ELISA of the anti-D monoclonal antibodies,
relative to Tegeline ®, with respect to several antigens

| | Degree of enrichment of the activity | | | | | | | |
|---|---|---|---|---|---|---|---|---|
| | relative to Tegeline ® | | | | | | | |
| Samples | Toxoid | DNP-Alb | DNA | F(ab)′2 | Actin | Myosin | MBP | Tubulin |
| D16/96 P1 | <0.1 | 2.7 | 4 | <0.1 | 1.86 | <0.1 | <0.1 | <0.1 |
| D39/96 P4 | <0.1 | 3.52 | 0 | <0.1 | 1.2 | <0.1 | <0.1 | <0.1 |
| D12/96 P5 | <0.1 | 2.28 | 0 | <0.1 | 3.1 | <0.1 | <0.1 | 1.69 |
| D12/96 P7 | <0.1 | 1.06 | 0 | <0.1 | 1.75 | <0.1 | <0.1 | <0.1 |
| D41/96P6/1 | <0.1 | 213 | 400 | <0.1 | 119 | <0.1 | <0.1 | 133 |
| D32/97 P1 | <0.1 | 154 | 3.4 | <0.1 | 2.36 | <0.1 | <0.1 | <0.1 |
| D49/97 P3 | <0.1 | 0.38 | 0.04 | <0.1 | 0.43 | <0.1 | <0.1 | <0.1 |
| D35/97 P3 | <0.1 | 0.03 | 0 | <0.1 | 0.08 | <0.1 | <0.1 | <0.1 |
| D83/97 P4 | <0.1 | 0.29 | 0 | <0.1 | 0.09 | <0.1 | 0.06 | <0.1 |
| Df5/97 P6 | <0.1 | 0.19 | 0 | <0.1 | 0.17 | <0.1 | 0.36 | <0.1 |
| D83/97 P7 | <0.1 | 3.45 | 0 | <0.1 | 0.24 | <0.1 | 0.51 | <0.1 |
| D31/97 P8 | <0.1 | 4.15 | 2.75 | <0.1 | 3 | <0.1 | 0.71 | 2.18 |
| Pf210 | 0.16 | 8.8 | 8.4 | NT | 2.8 | 2.5 | 0.15 | 6.1 |

The ability of the anti-D Mabs to inhibit the proliferation of human lymphocytes that are incompatible in MLC is greater than that of Tegeline® for all the antibodies, whether or not they react with DNP (FIG. 5).

The study of the release of cytokines into the supernatant of the MLCs shows that only the two Mabs D41 and D32 react with DNP, causing an increased synthesis of Il-1Ra which is proportional to their DNP reactivity (FIG. 6). The synthesis of other cytokines is either decreased (IL-4) or not modified (gIFN and TNFa) by the Mabs whatever their reactivity (FIG. 6).

The supernatant from the THP1 cells stimulated with the Mabs D41 and D32 contains a considerable amount of IL1-Ra, whereas, at the same concentrations, the Mabs D35 and D83 induce only a small production of this cytokine (FIG. 7).

### Example 2.2

### Production of Monoclonal Antibodies From Human Peripheral Blood Lymphocytes Originating From a Normal Donor

B lymphocytes originating from the blood of normal donors were transformed with EBV and placed in culture in a proportion of 10⁵ cells per well. The supernatants from the wells were tested for the presence of IgGs capable of recognizing the hapten DNP and actin, in an ELISA. The four most positive wells were pooled (C1) and cloned by dilution to 1000, 100, 50 and 10 cells per well. The same approach was used to select wells negative for anti-DNP and actin activity (C2) (table 4).

TABLE 4
Reactivity by ELISA of purified IgGs originating from culture supernatants
of EBV+ human B cells, with respect to several antigens

| | Degree of enrichment of the activity | | | | | | |
|---|---|---|---|---|---|---|---|
| | relative to Tegeline ® | | | | | | |
| Samples | Toxoid | DNP-Alb | F(ab)′2 | Actin | Myosin | MBP | Tubulin |
| Preparation 1 C1 | 1.2 | 40 | 6.1 | 16 | 126 | 27 | 56 |
| Preparation 2 | 5.8 | 115 | 19 | 96 | 392 | 276 | 212 |
| Preparation 1 C2 | 0 | 0.9 | 0 | 1.1 | 0 | 0 | 0 |
| Preparation 2 | 0.2 | 3.9 | 0.1 | 6.2 | 5 | 1.4 | 5.1 |

The cells of wells C1 and C2 were amplified so as to obtain amounts of antibodies sufficient to be purified on protein G. The antibodies obtained are of IgG type, and table 4 gives the results of two purifications carried out using supernatants taken on D+7 (preparation 1) and D+14 (preparation 2) and shows that the purified IgGs from well C1 recognize DNP and are reactive for a certain number of autoantigens. On the other hand, the IgGs from well C2 have no activity that is enriched relative to Tegeline®.

The IgGs purified from the two wells C1 and C2 were incubated at a dose of 25 μg/ml in the presence of THP1 cells. Cell growth is inhibited in the presence of the IgGs from C1, but not from C2 (data not shown), and only the IgGs from C1 stimulate the production of IL-1Ra by the THP1 cells, in vitro (FIG. 8).

The cells from wells C1 and C2 are in the process of being cloned at 0.5 cells per well in order to obtain monoclonal IgGs.

All these results show a positive correlation between the reactivity of IgG-type antibodies with respect to several autoantigens and the increased ability to induce the synthesis of IL-1Ra via monocytes or macrophages, whether the antibodies are polyclonal or monoclonal.

1. A monoclonal IgG class antibody which reacts with the hapten DNP and at least one autoantigen selected from myosin, actin, myelin basic protein (MBP) and tubulin, characterized in that it induces over-expression of IL-1Ra in macrophages or monocytes.

2. The antibody as claimed in claim 1, characterized in that it does not react with tetanus toxoid.

3-23. (canceled)
